(19) United States
(12) Patent Application Publication
(10) Pub. No.: US 2014/0161428 A1
(43) Pub. Date: Jun. 12, 2014

(54) Devices And Methods For Emanating Liquids

(71) Applicant: Helen Stephenson, Hull (GB)

(72) Inventor: Helen Stephenson, Hull (GB)

(73) Assignee: Reckitt & Colman (overseas) Limited, Slough, Berkshire (GB)

(21) Appl. No.: 14/127,711

(22) PCT Filed: Jun. 22, 2012
(86) PCT No.: PCT/GB2012/051455

§ 371 Date: Feb. 20, 2014

(30) Foreign Application Priority Data
Jun. 24, 2011 (GB) 1110694.5

(51) Int. Cl. A61L 9/03; A61L 9/12; A61L 9/14

(52) U.S. Cl. CPC A61L 9/037; A61L 9/14; A61L 9/127
USPC 392/395; 239/44; 239/145

(57) ABSTRACT

A liquid dispensing system comprising a universal refill of liquid and an emanation device; wherein the refill comprises: a housing; at least one reservoir in the housing for holding the liquid; at least one aperture in the housing sealed by at least one valve wherein said at least one valve defines a liquid pathway from the reservoir to the exterior of the refill, wherein at least one of said valve(s) is configured to be automatically resealable when not being held open; at least one wick housed entirely within the housing; and wherein the emanation device is selected from: an emanation device configured to spray the liquid; an emanation device configured to evaporate the liquid by electrically powered means; an emanation device configured to evaporate the liquid by passive, non-electrically powered means; an emanation device configured to emanate the liquid by two or more of spraying the liquid, evaporating the liquid by electrically powered means and/or evaporating the liquid by passive, non-electrically powered means; characterised in that each emanation device is provided with a chassis configured to receive and releasably retain the universal refill and the universal refill is operationally compatible with each emanation device.

### FIELD OF THE INVENTION

The present invention relates to devices and methods for improved airborne delivery of liquids containing one or more active materials wherein the active material comprises at least one of: a fragrance; an insecticide; a fungicide; a pesticide; a sanitising material; and/or a pharmaceutical.

### BACKGROUND

Liquids, and commonly volatile liquids, containing one or more active materials wherein the active material comprises at least one of: a fragrance; an insecticide; a fungicide; a pesticide; a sanitising material; and/or a pharmaceutical are delivered within the domestic environment via a variety of mechanisms. Devices are available with heaters disposed therein to increase the rate of emanation from a surface saturated with the liquid, such a surface could be a wick saturated with a fragranced liquid and the heater is located adjacent the wick surface and nearby a chimney to heat the liquid on the wick surface and cause it to more readily evaporate and disseminate into the surrounding environment through the chimney.

Alternatively such liquids can be loaded into an aerosol canister wherein the canister holds the liquid under pressure and when a valve is opened the liquid is forced out. The liquid is provided with a propellant which evaporates inside the canister to maintain an even pressure and, outside the canister, assist with the mechanical break up of the liquid by evaporating rapidly. Suitable propellants include volatile hydrocarbons such as propane, butane or isobutane.

Aerosols generally provide a satisfactory spray performance but since they require manual operation by a user, they are not considered to be particularly convenient for routine use.

Automatic aerosol activation devices exist for operation with metered dose aerosols containing a single phase liquid and propellant mixture. These devices are operable to periodically actuate the aerosol to cause a dose of the liquid to be sprayed.

Whilst the automation of the aerosol spraying devices overcomes the problem of manual operation of an aerosol canister, the use of aerosols containing such propellants is becoming increasingly less desirable since these volatile hydrocarbons carry the disadvantage of being flammable, the rising cost of oil is rendering them increasingly expensive and they also carry the further disadvantage of being an atmospheric source of carbon which is deemed to not be environmentally sensitive.

There are other known emanation devices using alternative emanation methodologies such as nebulisation techniques using a piezo-actuated apparatus for instance.

All of the known emanation devices and methodologies of emanation possess advantages and disadvantages with respect to each other that will be appreciated by a person skilled in the art as well as the ultimate end user. However, invariably the consumer is faced with a dilemma when purchasing any particular emanation device as the emanation method employed by the emanation device under consideration is not the only factor the consumer must also consider, the consumer must also consider the availability and range of refills of liquids to be used with said device. In the field of air freshening in particular where the range of fragrances the consumer can select for use with the device is a key point of purchase consideration for the consumer, a small range of fragrances and/or fear that the refill range may not be supported in the long term may affect the consumer's true freedom of choice. The present invention addresses this and other problems.

### SUMMARY OF INVENTION

According a first aspect of the present invention there is provided therefore a universal refill of liquid containing one or more active materials wherein the refill comprises:

a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing; and
wherein the refill is configured to allow the liquid to be extracted by at least two different methods of liquid extraction.

According a second aspect of the present invention there is provided therefore a universal refill containing a liquid for use with at least two different types of emanation device;

wherein the universal refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein each type of device emanates the liquid from the universal refill via a different mechanism to each other type of emanation device and wherein said at least two different types of emanation device each comprise a chassis configured to receive and releasably retain the universal refill.

According to a third aspect of the present invention there is provided a liquid dispensing system comprising a universal refill of liquid and an emanation device; wherein the refill comprises:

a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein the emanation device is selected from: an emanation device configured to spray the liquid; an emanation device configured to evaporate the liquid by electrically powered means; an emanation device configured to evaporate the liquid by passive, non-electrically powered means;
an emanation device configured to emanate the liquid by two or more of spraying the liquid, evaporating the liquid by electrically powered means and/or evaporating the liquid by passive, non-electrically powered means;
characterised in that each emanation device is provided with a chassis configured to receive and releasably retain the universal refill and the universal refill is operationally compatible with each emanation device.

According to a fourth aspect of the present invention there is provided a liquid dispensing system comprising a universal refill of liquid and an emanation device; wherein the refill comprises:

a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill and the universal refill is operationally compatible with each emanation device.

Within the context of the present invention the term “operationally compatible” is to be understood to require that the refill can be received within the chassis of an emanation device and permit the device to extract the liquid from the refill and emanate same into the surrounding environment.

According to a fifth aspect of the present invention there is provided a liquid dispensing device for use with a universal refill of liquid and an emanation device; wherein the refill comprises:

a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill.

Preferably the universal refill is provided with securing means which are configured, in use, to cooperate with the chassis and/or the emanation device to permit secure engagement therewith. Preferably the securing means form a part of the external surface of the housing.

The at least one valve may be provided by a single automatically resealable valve. The single valve may be provided in the form of a self-sealing liquid-tight valve, such as a silicone valve or the like. Alternatively the single valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In a preferred arrangement however, the or each aperture is provided with at least two valves. When at least two valves are provided a downstream valve (i.e. the valve closest to the reservoir) and an upstream valve is provided. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Whilst the annular ring is preferably provided in a circular shape other shapes may be permissible providing the shape is capable of performing the sealing function required of it, such shapes include substantially circular shapes, oval shapes, diamond shapes and such like.

The downstream valve is preferably operable to prevent any fluid within the refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In one preferred embodiment the downstream valve and the upstream valve are formed as separate pieces that are held adjacent but spaced apart from each other. In a most preferred embodiment however the downstream valve and the upstream valve are formed as a one piece component.

In a most preferred embodiment the refill is provided with a single aperture closed by a valve system comprising two valves wherein one of said valves is configured to be automatically resealable when not being held open and the other of said valves is provided in the form of an annular ring valve.

Advantageously the valve system(s) of the present invention may ameliorate, and for some types of liquid substantially completely remove, the damaging effects of fractionation as all the liquid and any naturally volatilised quantity thereof are retained within the refill until extraction is required.

Said at least one valve could be adjustable to affect the flow rate of the liquid therethrough. The adjustability may be facilitated manually by a user and/or due to an automated function of a device to which the refill is connected and said automated function may be controlled automatically by the device or may be in response to a user input into the device. Adjustability may be particularly useful when the device is configured to emanate the liquid passively and/or extract the liquid passively such as by gravity feed.

Preferably the universal refill is provided with one or more diptubes therein.

The universal refill is preferably formed with a housing of rigid or substantially rigid material. Such an arrangement may be advantageous as differing forms of extraction of liquid therefrom may put different strains and pressures on the refill and by providing the housing of the refill in a rigid or substantially rigid material, the refill may be better able to resist those differing strains and pressures to deliver uniform and/or predictable characteristics.

The refill housing is preferably shaped such that a user may only able to engage the refill with a chassis in a single orientation. For instance, if the refill was generally cylindrical with the valve system(s) being provided on the circular end of the cylinder, a user could load the circular end into the chassis in various orientations which may prevent ease of engagement of the refill therein, whereas if the face of the refill containing the valve system(s) was, for instance, rhomboidal then the user would be presented with only a single option for orienting the refill in the chassis.

Alternatively, the refill housing may be shaped such that a user may ostensibly be presented with more than one orientation in which they could engage the refill with a chassis and in this arrangement the chassis is preferably provided with guide means which cooperate with features of the refill housing to ensure that the refill can only be engaged with the chassis in a single orientation such that efforts by the user to incorrectly load the refill in the chassis will be resisted thereby.

The universal refill is preferably provided with a single reservoir. The presence of a diptube may be advantageous as this may better optimise the ability of the universal refill to operate in a number of different orientations when engaged with the chassis of an emanation device. The diptube may be connected to the valve system or may be held adjacent to the valve system by mounting fixed within the refill.

The reservoir(s) may be provided in the form of a flexible bag which is capable of collapsing inwardly as liquid is extracted therefrom.

The refill may be provided with one or more vent holes in the refill housing which are formed to permit air to enter the reservoir(s) when liquid is removed therefrom but not to permit the liquid to escape therethrough. The vent hole(s) may be covered with a gas permeable membrane. Alternatively the vent hole(s) may be covered with an automatically resealable means which engages with a seal opening means of the chassis. Preferably however, the vent hole(s) are preferably sized such that the liquid may not escape therefrom or that the rate of escape would be sufficiently low as to not be relevant for the safe operation of the refill in an emanation device.

The refill housing is preferably provided with a section thereof adjacent the reservoir(s) that is substantially transparent to permit a user to easily determine what type of liquid is held therein and/or the level of said liquid.

The chassis may be formed as a separate component to the emanation device and may be mounted on or in the device to receive and releasably retain the universal refill. Alternatively or additionally, the chassis may be formed in an exterior surface of the emanation device (such as a device housing). Alternatively or additionally the chassis may be formed within the interior of the device and a suitably shaped aperture is provided to permit access to the interior of the housing by the universal refill. Alternatively or additionally the chassis may be formed within the interior of the device and the device is provided with a door which may be opened to permit access to the interior of the housing by the universal refill.

Where the emanation device is to pump the liquid from the reservoir(s) of the universal refill the emanation device may be provided with one or more liquid conduits that are connected at one end to a pumping means housed within the device and open at the other end to contact the liquid. The liquid conduit(s) are preferably sized to extend through the, or each, valve system in the universal refill and into the reservoir(s) therewithin when the refill is engaged with the chassis. In use, the pump may be operable via conventional pumping mechanisms to create negative pressure within the liquid conduit(s) in order to draw the liquid into the conduit(s) and transport same toward the pump. Once the liquid is in the device it may be emanated therefrom in any of a number of convention mechanisms, for instance the pumped liquid may be transported to an open emanation plate in combination with a heater to promote evaporation of the liquid, or the liquid may be transported to a nebuliser and it is atomised thereby, etc.

Where the emanation device is to blow the liquid from the reservoir(s) of the universal refill the emanation device may be provided with one or more blown air conduits that are connected at one end to a blowing means housed within the device and open at the other end to permit blown air to exit therefrom and with one or more liquid conduits that are open at one end to contact the liquid and connected to the device at the other end. The blown air conduit(s) are preferably sized to enter through one valve system of the universal refill and the liquid conduit(s) are preferably sized to extend through one or more of alternate valve systems in the refill (i.e.—the blown air conduit(s) and the liquid conduit(s) do not use the same valve system) and wherein both types of conduits extend into the reservoir(s) within the refill when said refill is engaged with the chassis. In use, the blowing means may be operable to blow air along the blown air conduit and into the reservoir(s) in order to pressurise the head space (i.e.—the head space is the space within the reservoir(s) not taken up by the liquid) and, when required by the device, force liquid into the liquid conduit(s) and toward the device. liquid transported along the liquid conduit(s) may be emanated therefrom in any of a number of convention mechanisms, for instance the liquid may be sprayed directly from the device under the force of said transportation and/or in combination with additional blown air from the blowing means, the liquid may be transported to a nebuliser and for it to be atomised thereby, etc.

Alternatively, where the universal refill is provided with a single aperture and single valve system, the blown air conduit and liquid conduit could be integrated such that the blown air conduit extends along a central core of the liquid conduit with the blown air conduit being provided with the greater length such that it can extend further into the refill and such that the conduits can enter the single aperture and valve system; the blown air conduit could be adjacent the liquid conduit however. In this arrangement the blown air conduit may act as described above by blowing air into the headspace of the refill to force liquid into the liquid conduit and into the device for emanation therefrom. As a further alternative, an integrated conduit can be provided with one conduit extending along a central core of the other conduit such that the conduits can both enter a single aperture and valve system, wherein the one conduit is a liquid conduit which has air blown across the top thereof to draw the liquid along the conduit via the Venturi effect and/or capillary effect, whilst the other conduit allows air to be returned to the reservoir to prevent choking of the liquid flow.

Where the emanation device is to wick the liquid from the reservoir(s) of the universal refill the emanation device may be provided with one or more wicks that are connected at one end to the emanation device and configured at the other end to contact the liquid and uptake same. The wick(s) are preferably sized to extend through the valve system(s) in the universal refill and into the reservoir(s) therewithin when the refill is engaged with the chassis. In use, the wick(s) may be operable to passively uptake liquid from the reservoir(s) and transport same towards the other end of the wick(s). Once the liquid is within the wick(s) may be emanated from the device in any of a number of convention mechanisms, for instance the liquid may be emanated directly from the wick(s) passively and/or in combination with powered means to assist in the evaporation such as an electrically powered heater, or the liquid may be transported to along the wick(s) to a nebuliser and it is atomised thereby, etc.

An alternative preferred embodiment for when the emanation device is to wick the liquid from the reservoir is to rely on the wick housed entirely within the refill. In this preferred embodiment the emanation device may be provided with an extraction limb that is configured to heat up in use to impart heat within the interior of the universal refill. The extraction limb may be hollow and elongate and possess at least one aperture therein to define a fluid flow path from the said at least one aperture into the hollow extraction limb and to said at least one exit port of the emanation device.

Where the emanation device is to transport the liquid from the reservoir(s) of the universal refill to the emanation device via capillary action the device may be provided with one or more capillary tubes that are connected at one end to the emanation device and configured at the other end to contact the liquid and uptake same. The capillary tube(s) are preferably sized to extend through the valve system(s) in the universal refill and into the reservoir(s) therewithin when the refill is engaged with the chassis. In use, the capillary tube (s) may be operable to passively uptake liquid from the reservoir(s) and transport same towards the other end of the tube(s). liquid transported along the capillary tube(s) to the emanation device may then be emanated therefrom in any of a number of convention mechanisms, for instance the liquid may be transported to an open emanation plate in combination with a heater to promote evaporation of the liquid, or the liquid may be transported to a nebuliser and it is atomised thereby, etc.

Where the emanation device is to extract the liquid from the reservoir(s) of the universal refill toward the emanation device by a gravity feed mechanism the emanation device may be provided with one or more liquid conduits that are connected at one end to the device and open at the other end to contact the liquid. The liquid conduit(s) are preferably sized to extend through the valve system(s) in the universal refill and into the reservoir(s) therewithin when the refill is engaged with the chassis. The chassis is orientated such when the universal refill is engaged therewith the valve system(s) of the refill are located closer to a supporting surface on which the device is held such that once the valve(s) are opened by the liquid conduit(s) the force of gravity will cause liquid to travel from the refill toward the device. In use, the liquid conduit(s) may be operable to simply open the valve system(s) to permit the liquid to flow from the reservoir toward the device to emanate the liquid by any of a number of convention emanation mechanisms, for instance the liquid flow into an open emanation plate in combination with a heater to promote evaporation of the liquid, or the liquid may flow toward a nebuliser for it to be atomised thereby, etc.

The emanation device and/or chassis may be provided with securing means which are configured to engage with the universal refill to permit a secure engagement of the universal refill to the chassis. Preferably the releasable engagement between the universal refill and the chassis is provided with a positive feedback for the user; such as a “push-click” mechanism wherein the user pushes the refill against the chassis until an audible “click” sound is achieved and the refill is secured relative to the chassis, and to disengage the universal refill from the chassis the user pushes the refill against the chassis until a further “click” sound is achieved accompanied by a physical sensation of the refill being pushed away from the chassis and the user may separate the refill from the device.

The universal refill and emanation devices are preferably provided with a device-to-refill recognition system whereby the device is able to recognise the level of liquid remaining in the refill and/or the type of liquid in the refill and/or whether the refill is from a known source. The recognition system may be provided by the device possessing a physical characteristic in the refill housing which interacts with a portion of the chassis and/or device. The recognition system may comprise a moulded feature in the reservoir(s) which may have a communication component embedded therein which can communicate with a corresponding communication component within the device and/or the moulded feature may act as a convenient means to for the device to make reference against when determining the level of liquid in the reservoir(s). The recognition system may be provided with an identification means being applied to and/or in the refill housing such as a visual code, an RFID tag, printed electrical components, magnets or the like. The recognition system may be provided by any combination of the aforesaid recognition systems.

Any of the features described herein may be combined with any of the above aspects in any combination.

### BRIEF DESCRIPTION OF THE DRAWINGS

Embodiments of the invention will now be described, by way of example only, with reference to the following drawings in which:

FIG. 1 shows a perspective view of the refill;

FIG. 2 shows a perspective view of an extraction limb of an emanation device;

FIG. 3 shows a sectional view of the refill valve;

FIG. 4 shows a sectional view of the refill with a mechanism for a gravity-feed extraction emanation device;

FIG. 5 shows a sectional view of the refill with a mechanism for a heated extraction emanation device; and

FIG. 6 shows a sectional view of the refill with a mechanism for a blown air extraction emanation device.

### DESCRIPTION OF AN EMBODIMENT

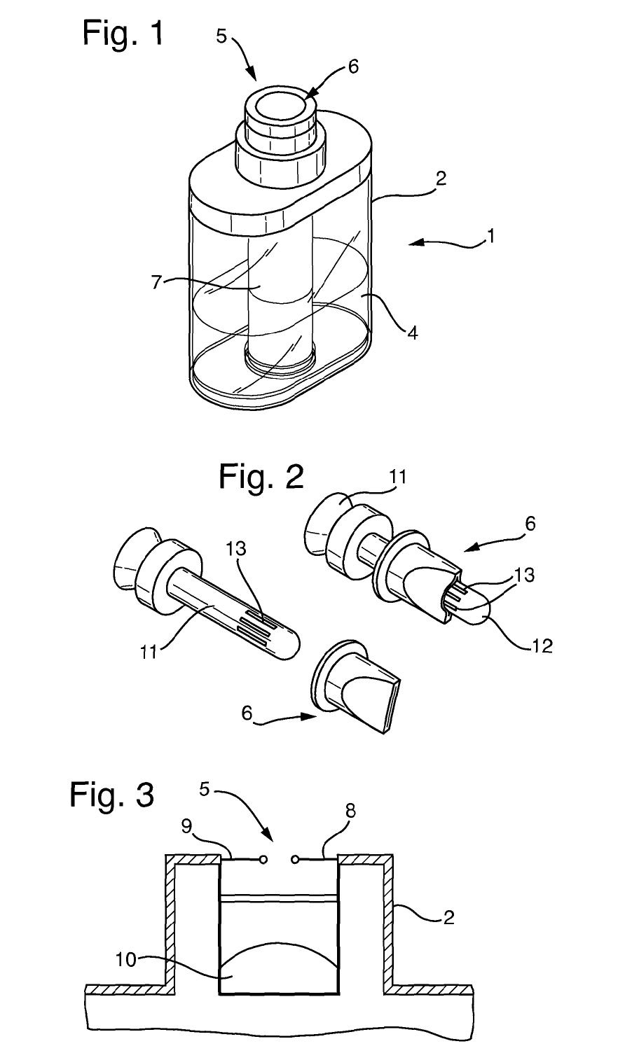

FIG. 1 shows a preferred embodiment of a refill 1 according to the present invention. The refill 1 comprises a housing 2 that surrounds and contains a reservoir 3 of liquid 4 therein. Access to the interior of the refill 1 and the reservoir 3 is provided via aperture 5 which is sealed by a valve 6. Housed entirely within the housing 2 of the refill 1 is a wick 7. The wick 7 is shown in FIG. 1 as a having a substantially cylindrical shape that extends from adjacent the valve 5 to contact a base of the reservoir 3.

Although not shown in FIG. 1, when the valve 6 is held in an open position, a notional elongate column of space within the interior of the housing extending from the perimeter of the valve opening to the lower wall of the housing is defined, hereinafter referred to as the elongate column. The wick 7 has a cylindrical shape that is hollow to substantially surrounds the elongate column.

FIG. 3 shows the valve 6 is greater detail. The valve 6 is an automatically resealable valve system comprising an open annular ring valve 8 suspended by a flexible annular skirt 9 which forms the upstream valve and the valve system further comprises a downstream valve in the form of a rubberised slit valve 10. The downstream slit valve 10 provides a fluid tight closure to prevent the liquid in the reservoir from escaping.

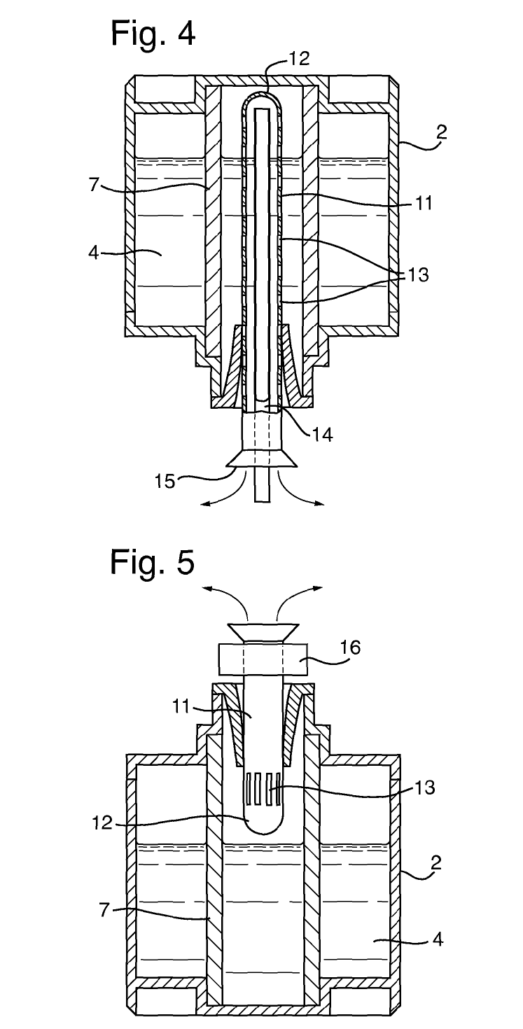

FIG. 4 shows how an emanation device can interact with the valve system of the refill 1. A hollow extraction limb 11 having a rounded end section 12 may be provided operatively connected to the emanation device. The limb 11 is provided with a series of perforations 13 which provide access to the hollow interior of the limb 11. Preferably the limb 8 is sized to have a diameter that is slightly wider than the diameter of the ring valve 8 such that, in use, when the rounded end 12 is pushed through the ring valve 8 a liquid-tight connection is made between the limb 11 and the ring valve 8. On travelling further into the valve 6 the rounded end will meet the slit valve 10 and force it open. Since the limb 11 and the ring valve 8 have formed a liquid-tight connection any liquid that is not able to flow past the slit valve 10 will be prevented from leaking out of the refill 1. The limb 11 will be pushed into the valve 5 such that the perforations 13 also pass the slit valve 10 to be disposed within the interior of the refill 1.

A mechanism that is suitable for a gravity-fed emanation device is shown in FIG. 4. In this arrangement the refill 1 is loaded into the device in an inverted orientation such that the extraction limb 11 is generally upstanding and the valve 5 of the refill is generally the lowest part thereof. In this arrangement it can be seen that the extraction limb comprises a hollow limb with perforations 13 and further comprising a central hollow core 14 without perforations but with open ends. To extract the liquid 4 from the refill 1 in this arrangement, the refill 1 is loaded on to the extraction limb 11 to form a liquid-tight seal between the limb 11 and the ring valve 8. Liquid is permitted to enter the hollow interior of the limb 11 via the perforations 13 but the liquid is not present in a sufficient volume to enter the open end of the central hollow core 14. When the device is activated the liquid may flow in a downward direction powered by gravity and toward an open end 15 of the limb 11 and into the device. The central hollow core 14 may be open to the atmosphere or connected to an air pump in order to return air to the reservoir 3 of the refill 1 to prevent negative pressure from developing within the refill 1 and retarding or choking the flow of liquid 4 into the device.

A mechanism that is suitable for a heated emanation device is shown in FIG. 5. In this arrangement the refill 1 is loaded into the device in an upright orientation such that the valve 5 is generally the uppermost part of the refill 1. Again the refill 1 is loaded on to the extraction limb 11 to form a liquid-tight seal between the limb 11 and the ring valve 8, and preferably the limb enters the refill such that the perforations 13 are below the slit valve but also so that the rounded end 12 is not in contact with the liquid 4. The extraction limb 11 is provided with a heater 16 connected thereto. The heater 16 is configured to heat the extraction limb 11, the limb 11 being made from a suitably heat-conductive material, in order to transmit heat to the interior of the refill 1. The heater 16 is configured to transmit enough heat to the interior of the refill 1 to volatilise the liquid held in the wick 7, the resulting volatilised liquid may then enter the perforations 13 and travel upwardly into the device and out into the surrounding environment.

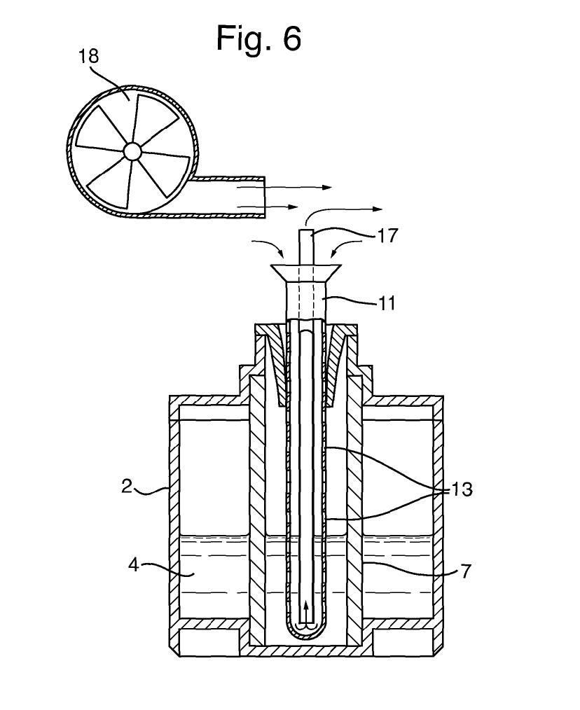

A mechanism that is suitable for a heated emanation device is shown in FIG. 6. In this arrangement the refill 1 is loaded into the device in an upright orientation such that the valve 5 is generally the uppermost part of the refill 1. The refill 1 is loaded on to the extraction limb 11 to form a liquid-tight seal between the limb 11 and the ring valve 8, and preferably the limb enters the refill such that the perforations 13 are below the slit valve and also below the liquid level, to this end the perforations 13 may be adjacent to the rounded end 12. The limb 11 may also be provided with a central hollow liquid conduit 17 that is open at both ends. The upper end of the conduit 17 may be adjacent a an air pump 18 that is configured to blow air across the top of the conduit 17 to cause liquid to travel up the conduit by both capillary action and the Venturi effect and be blown into the surrounding environment. In this arrangement air would be permitted to be returned into the reservoir 3 to prevent negative pressure from developing within the refill 1 and retarding or choking the flow of liquid 4 into the device by air travelling within the hollow limb 11 between an inner surface of the limb and an outer surface of the conduit and passing into the reservoir through the perforations.

Although not shown, the end of the housing 1 containing the valve 6 may be substantially square in shape and guide means (not shown) in a chassis (not shown) of each emanation device) could cooperate with grooves in the refill housing (not shown) to ensure that the refill could only be engaged with the chassis if the limb 11 is correctly aligned to open valve 6. Although not illustrated, the end of the refill housing containing the valve 6 could be shaped such that the loading thereof into the chassis was only possible via a single orientation of that end of the refill.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A liquid dispensing system comprising a universal refill of liquid and an emanation device; wherein the refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein the emanation device is selected from: an emanation device configured to spray the liquid; an emanation device configured to evaporate the liquid by electrically powered means; an emanation device configured to evaporate the liquid by passive, non-electrically powered means; an emanation device configured to emanate the liquid by two or more of spraying the liquid, evaporating the liquid by electrically powered means and/or evaporating the liquid by passive, non-electrically powered means;
characterised in that each emanation device is provided with a chassis configured to receive and releasably retain the universal refill and the universal refill is operationally compatible with each emanation device.

2. A liquid dispensing system comprising a universal refill of liquid and an emanation device; wherein the refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein the emanation device is selected from: an emanation device configured to extract the liquid from the refill by pumping the liquid therefrom;
an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill and the universal refill is operationally compatible with each emanation device.

3. A liquid dispensing device for use with a universal refill of liquid and an emanation device; wherein the refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
wherein the emanation device is selected from: an emanation device configured to extract the liquid from the refill by pumping the liquid therefrom; an emanation device configured to extract the liquid from the refill by blowing the liquid therefrom; an emanation device configured to extract the liquid from the refill by wicking the liquid therefrom; an emanation device configured to extract the liquid from the refill by capillary action therefrom; an emanation device configured to extract the liquid from the refill by a gravity feed therefrom; an emanation device configured to extract the liquid from the refill by two or more ways selected from pumping, blowing, wicking, capillary action and/or gravity feed therefrom;
characterised in that each emanation device is provided with a chassis configured to receive and releasably retain the universal refill.

4. A liquid dispensing system according to claim 1, wherein the universal refill is provided with securing means which are configured, in use, to cooperate with the chassis and/or the emanation device to permit secure engagement therewith.

5. A liquid dispensing system according to claim 1, wherein each aperture is provided with at least two valves.

6. A liquid dispensing system according to claim 5, wherein the at least two valves are provided as a downstream valve and an upstream valve.

7. A liquid dispensing system according to claim 6, wherein the downstream valve is provided by a self-sealing liquid-tight valve wherein the valve tends toward a closed position when not being held open, or provided by a sealing means biased into a closed position by a deformable biasing means such as a spring means or the like.

8. A liquid dispensing system according to claim 6, wherein the upstream valve is provided as an annular ring which is an open ring that is not able to prevent fluid flow therethrough.

9. A liquid dispensing system according to claim 8, wherein the annular ring is supported on a flexible annular skirt.

10. A liquid dispensing system according to claim 1, whereby when the emanation device is to pump the liquid from the reservoir(s) of the universal refill, the emanation device is provided with one or more liquid conduits that are connected at one end to a pumping means housed within the device and open at the other end to contact the liquid.

11. A liquid dispensing system according to claim 1, whereby when the emanation device is to blow the liquid from the reservoir(s) of the universal refill the emanation device is provided with one or more blown air conduits that are connected at one end to a blowing means housed within the device and open at the other end to permit blown air to exit therefrom and with one or more liquid conduits that are open at one end to contact the liquid and connected to the device at the other end.

12. A liquid dispensing system according to claim 1, whereby when the emanation device is to wick the liquid from the reservoir(s) of the universal refill the emanation device is provided with an extraction limb that is configured to heat up in use to impart heat within the interior of the universal refill.

13. A liquid dispensing system according to claim 12, wherein the extraction limb is hollow and elongate and possess at least one aperture therein to define a fluid flow path from the said at least one aperture into the hollow extraction limb and to said at least one exit port of the emanation device.

14. A liquid dispensing system according to claim 1, whereby when the emanation device is to transport the liquid from the reservoir(s) of the universal refill to the emanation device via capillary action the device is provided with one or more capillary tubes that are connected at one end to the emanation device and configured at the other end to contact the liquid and uptake same.

15. A liquid dispensing system according to claim 1, whereby when the emanation device is to extract the liquid from the reservoir(s) of the universal refill toward the emanation device by a gravity feed mechanism the emanation device is provided with one or more liquid conduits that are connected at one end to the device and open at the other end to contact the liquid.

16. A liquid dispensing system according to claim 1, wherein at least one of the emanation device and/or chassis are provided with securing means which are configured to engage with the universal refill to permit a secure engagement of the universal refill to the chassis.

17. A universal refill of liquid containing one or more active materials wherein the refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing; and
wherein the refill is configured to allow the liquid to be extracted by at least two different methods of liquid extraction.

18. A universal refill containing a liquid for use with at least two different types of emanation device;
wherein the universal refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
at least one wick housed entirely within the housing;
and wherein each type of device emanates the liquid from the universal refill via a different mechanism to each other type of emanation device and wherein said at least two different types of emanation device each comprise a chassis configured to receive and releasably retain the universal refill.
